(19) United States
(12) Patent Application Publication
(10) Pub. No.: US 2002/0188408 A1
(43) Pub. Date: Dec. 12, 2002

(54) Clearinghouse methods and systems for processing bioinformatic data

(76) Inventor: Antoun Alexander Nabhan, Cambridge, MA (US)

Correspondence Address: MYERS BIGEL SIBLEY & SAJOVEC, PO BOX 37428, RALEIGH, NC, 27627, US

(21) Appl. No.: 09/876,369

(22) Filed: Jun. 7, 2001

(51) Int. Cl. G06F 17/60; G06F 19/00; G01N 33/48; G01N 33/50

(52) U.S. Cl. 702/19; 702/20; 705/3

(57) ABSTRACT

Bioinformatic data is accepted from corresponding bioinformatic data suppliers. A subset of the bioinformatic data is analyzed to generate bioinformatic data analysis results. The bioinformatic data analysis results are provided to at least one bioinformatic data analysis results customer. The bioinformatic data suppliers that supplied the subset of the bioinformatic data are compensated in return for their supplying the subset of the bioinformatic data that was analyzed to generate the bioinformatic data analysis results that were provided to the at least one bioinformatic data analysis results customer.

### FIELD OF THE INVENTION

The present invention relates to bioinformatics, and more particularly to systems, methods and computer program products for processing bioinformatic data.

### BACKGROUND OF THE INVENTION

The sequence of the human genome can provide a valuable medical resource. Unfortunately, in order to use this vast amount of sequence information to develop new medical applications, a more sophisticated understanding of gene function may be needed. In a sense, genome sequencing efforts are yielding a large quantity of nouns, with the verbs and grammar yet to be fully discovered. Accordingly, much research effort has been focused on the interpretation of this vast amount of sequence information. This can result in a better understanding of the roles that genes and proteins play in biochemical pathways, and can thereby provide an understanding of the mechanisms of disease.

These advances in bioinformatics may also allow the drug discovery process to be transformed through rapid and efficient discovery of new drug targets in model organisms and human cells. In particular, drugs may target proteins or other compounds within each cell that are known to play a part in the biochemical pathway of a disease. When these targets are identified, users may test many compounds against them. Based on the reaction of the target to the compound, a determination may be made as to whether a potential drug candidate is likely to be successful.

Thus, bioinformatics has given rise to a variety of methodologies that are being used to discover new target molecules and therapeutic approaches. For example, the discovery of new targets may be facilitated by comparing the DNA sequence of the potential target with that of known targets. If the DNA is similar, the proteins which result also may be similar, suggesting that they will respond similarly to therapies. This approach also may be used to identify which molecular target in humans is likely to be analogous to a target previously identified in an animal model. Users also can identify targets by determining which genes are responsible for a given disease.

Bioinformatics also can identify genetic variations which are a major component, either as a cause or as an effect, of diseases, such as cancer, diabetes and cardiovascular disease. Disease risks can be identified by monitoring variations in responsible genes. This may be done by analyzing mutations of a single nucleotide base, referred to as a Single Nucleotide Polymorphism (SNP). Unfortunately, although SNPs may potentially indicate which drug will be best for a given individual, SNP analysis may need large-scale human studies to establish these useful associations. This may make SNP an expensive and difficult process, which also may be inaccurate, non-automated, inflexible and/or slow, depending on the implementation.

Bioinformatics companies may focus on generating large amounts of DNA sequence data. Unfortunately, without knowledge of the gene's functions, the DNA sequence data for a gene may be insufficient to materially impact the drug development process. Moreover, associations between DNA sequence and detailed cellular function may be complex, and may be generally unknown. Accordingly, detailed measurements of the actual biological functioning of the cell at a molecular level may be important to identify the best targets and illuminate mechanisms of disease.

Many approaches have been developed that can address these needs by monitoring changes in the levels of certain cellular components. One approach, referred to as expression profiling, monitors the level of messenger RNA (mRNA) for each gene within a cell. Expression profiling technologies can monitor tens of thousands of genes. Monitoring of tens of thousands of genes may be performed by arranging shorter, single-stranded DNA pieces, called oligonucleotides, in a dense grid on a substrate, such as a glass surface. This grid is known as a microarray. An oligonucleotide in a microarray may bind to the mRNA of a specific gene, to thereby provide an indication of that gene's expression level.

A second approach, referred to as “proteomics”, monitors the level of protein expressed by each gene within a cell. Proteomics measurements may be obtained by fractionating a mix of proteins in a cell, by separating the proteins through a resistive substance, such as a gel, so that proteins of different sizes and properties separate to different spots on the gel. This array of spots is analyzed, to thereby allow the monitoring of protein levels within the cell.

In view of the above, many independent organizations in the commercial, academic and governmental environments are involved in generating large quantities of bioinformatic data. Some of this data may be made publicly available. However, much of this data is maintained as proprietary data. Thus, discoveries that might be made by combining data that are by themselves inconclusive may not be made. For example, one organization might know, but keep private, the knowledge of a chromosomal proximity in mice between a gene of (privately) known function and one of unknown function. Another organization might know, but keep private, the knowledge of a chromosomal proximity in humans between a gene of (privately) known function and one of suspected function and with structural homology to the gene of unknown function in mice. Because locational proximity tends to correspond with functional similarity, a combination of these data might lend more certainty to a researcher's hypothesis regarding the function in humans of the suspected gene. Although there is often discussion within the bioinformatics community of sharing bioinformatic data for the overall benefit of science and humankind, there may be little economic incentive to do so. In fact, there may be economic disincentives in sharing this data.

### SUMMARY OF THE INVENTION

Embodiments of the present invention provide clearinghouse methods and systems for processing bioinformatic data. According to embodiments of the present invention, bioinformatic data is accepted from corresponding bioinformatic data suppliers. A subset of the bioinformatic data is analyzed to generate bioinformatic data analysis results. The bioinformatic data analysis results are provided to at least one bioinformatic data analysis results customer. The bioinformatic data suppliers that supplied the subset of the bioinformatic data are compensated in return for their supplying the subset of the bioinformatic data that was analyzed to generate the bioinformatic data analysis results that were provided to the at least one bioinformatic data analysis results customer.

Accordingly, bioinformatic data suppliers may be economically encouraged to contribute their bioinformatic data to the clearinghouse. The clearinghouse can perform value-added processing by combining bioinformatic data from multiple suppliers, to produce new bioinformatic data analysis results. A bioinformatic data analysis results customer can obtain value-added bioinformatic data analysis results. The bioinformatic data suppliers can benefit by being compensated based on their contribution to the value-added bioinformatic data analysis results that were sold.

Embodiments of the present invention, therefore, may provide incentives to bioinformatic data suppliers to contribute their data to a clearinghouse rather than maintaining the data as proprietary information. Bioinformatic data analysis results customers also may be encouraged to pay for the results, because the value-added results can be more valuable than those that may be obtained by analyzing bioinformatic data from a single supplier and/or internally generated proprietary data. The clearinghouse can retain a portion of the compensation that is received from the bioinformatic data analysis results customers as compensation for the clearinghouse's value-added data analysis and for acting as a clearinghouse. Multiple economic incentives thereby may be created that can encourage the sharing of bioinformatic data, for the potential benefit of science and humankind.

### BRIEF DESCRIPTION OF THE DRAWINGS

FIG. 1 is a block diagram of clearinghouse methods and systems for processing bioinformatic data according to embodiments of the present invention.

FIGS. 2-5 are flowcharts of operations that may be performed by clearinghouse methods and systems for processing bioinformatic data according to embodiments of the present invention.

FIG. 6 is an example of a bioinformatic data file according to embodiments of the present invention.

FIG. 7 is an example of a bioinformatic data object according to embodiments of the present invention.

### DETAILED DESCRIPTION OF PREFERRED EMBODIMENTS

As used herein, the following terms have the following meanings:

Bioinformatic Data—Information on the structure or function of an organism or a means of altering the state of an organism, including but not limited to genomic data, chemical compositions and effects of drugs and other therapies, medical patient data, and information about phenotypes or disease states.

Bioinformatic Data Analysis Results—the value-added results of analysis of bioinformatic data including information about causal relationships between genes, RNA, proteins and/or phenotypes or diseases. Examples include previously unknown specifications of biological pathways, previously unknown relationships between the expression patterns of multiple genes, gene sequences for genes that are discovered to be related in a particular biological phenomenon, peptide sequences for proteins that are discovered to be related to a pharmaceutically interesting biological phenomena, and/or the chemical specification of a binding site to a protein that is discovered to be related to a pharmaceutically interesting biological phenomena.

Bioinformatic Data Analysis Results Customers—commercial, academic or governmental entities that may use bioinformatic data analysis results, including large pharmaceutical companies, drug development companies, academic laboratories, medical doctors and/or genetic counselors. An entity may be both a bioinformatic data supplier and a bioinformatic data analysis results customer.

Bioinformatic Data Supplier—a commercial, academic or governmental entity, such as pharmaceutical company research and development labs, expression analysis outsourcers, genome sequencing centers and academic research laboratories.

Chloroplastic DNA—the DNA which resides in the chloroplast.

DNA—a molecule consisting of deoxyribonucleic acid sequences. Examples include cDNA, oligonucleotides, genomic DNA, mitochondrial DNA, chloroplastic DNA, plasmids and other forms of extrachromosomal DNA.

Gene—the functional unit of heredity. Each gene occupies a specific place (or locus) on a chromosome, is capable of reproducing itself exactly at each cell division, and is capable of directing the formation of an RNA and protein. The gene as a functional unit may consist of a discrete segment of a DNA molecule containing the proper number of purine (adenine and guanine) and pyrimidine (cytosine and thymine) bases in the correct sequence to code the sequence of amino acids needed to form a specific peptide.

Gene Expression—the active transcription of a gene into an RNA molecule and translation into protein, but also in the context of a particular tissue, the state of development or combinations of translated proteins.

Gene Expression Profile—the representation of genes that are being transcribed from the DNA and translated into proteins, but also in reference to a particular tissue, stage of development or combinations thereof.

Gene Expression Signature—summary of gene expression at one time in one profile—usually used in reference to pathology, but also in reference to the developmental stage of the organism, a response to stimuli such as drugs or environmental factors, tissue specificity, age, and/or disease progression.

Genome—The total gene complement of a set of chromosomes found in higher life forms; or, the functionally similar, but simpler, linear arrangements found in bacteria and viruses. A genome may include, or be represented as, genomic DNA or cDNA and also may include mitochondrial and chloroplastic DNA.

Genomic Data—information on some or all of a genome, including but not limited to gene expression, protein level, sequence and/or pathology data.

Genomic DNA—the DNA which makes up the entire chromosomal DNA of a life form.

Mitochondrial DNA—the DNA which resides in the mitochondria.

Pathology—the interpretation of diseases in terms of cellular operations; i.e. the way in which cells and cellular processes deviate from the homeostatic state.

Pathway—any sequence of chemical reactions leading from one compound to another.

Protein—a macromolecule consisting of sequences of alpha-amino acids in peptide linkage involved in structures, hormones, enzymes, and essential life functions.

RNA—a macromolecule consisting of ribonucleic acid sequences. Examples include viral RNA sequences, symptomless viral RNA sequences, ribozymes, mRNA, rRNA, tRNA and snRNA.

Structure—a tissue or formation made up of different or related parts; or, the specific connections of the atoms in a given molecule. Examples include muscle, nerve, skin, lung, liver, leaf, root, flower, stem and other tissues.

Other terms that are used herein are well known to those having skill in the art and need not be described in detail herein, or will be defined as they are used herein.

The present invention now will be described more fully hereinafter with reference to the accompanying drawings, in which preferred embodiments of the present invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

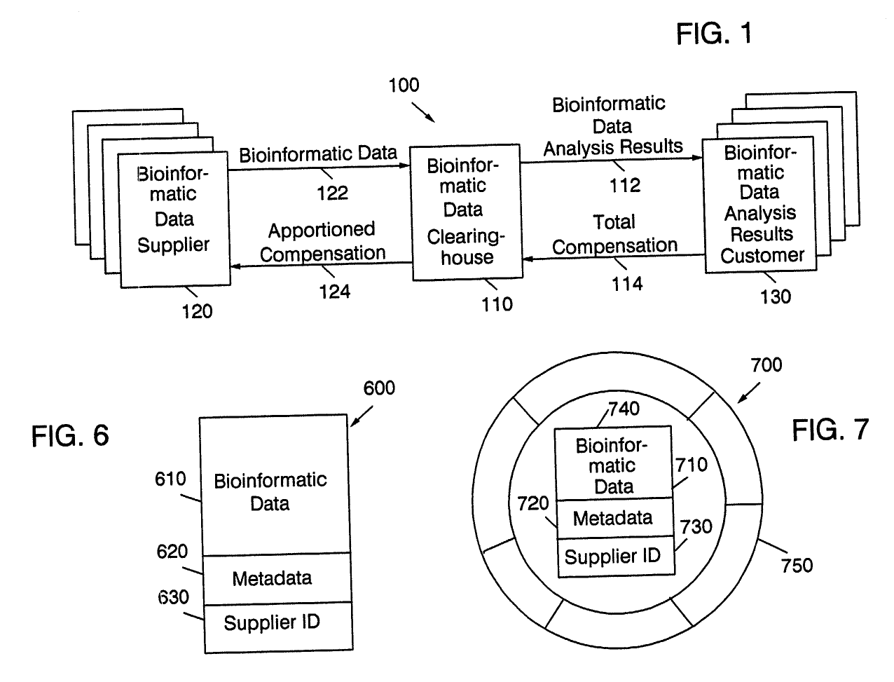

FIG. 1 is a block diagram of clearinghouse methods and systems for processing bioinformatic data according to embodiments of the present invention. As shown in FIG. 1, these clearinghouse methods and systems 100 include a plurality of bioinformatic data suppliers 120 that supply bioinformatic data 122 to a bioinformatic data clearinghouse 110. The bioinformatic data clearinghouse 110 is configured to accept the bioinformatic data 122 from the plurality of bioinformatic data suppliers 120, to analyze a subset of the bioinformatic data 120 to generate bioinformatic data analysis results 112, and to provide the bioinformatic data analysis results 112 to at least one bioinformatic data analysis results customer 130.

The bioinformatic data clearinghouse 110 also is configured to compensate, or authorize compensation for, the bioinformatic data suppliers 120 that supplied the subset of the bioinformatic data 122 for their supplying the subsets of the bioinformatic data that were analyzed to generate the bioinformatic data analysis results 112 that were provided to the at least one bioinformatic data analysis results customer 130. More specifically, as shown in FIG. 1, the bioinformatic data analysis results customers 130 supply a total compensation 114 such as a lump sum and/or royalty stream to the clearinghouse 110 as payment for the bioinformatic data analysis results 112. In other alternatives, non-monetary compensation 114 may be provided such as additional bioinformatic data, an equity interest and/or other value. Accordingly, as used herein, the term compensation can include any item of value that is provided by a bioinformatic data analysis results customer to the bioinformatic data clearinghouse. The clearinghouse 110 apportions compensation to the bioinformatic data suppliers 120 based on the contribution of the subset of the bioinformatic data to the bioinformatic data analysis results 112, and provides apportioned compensation 124 to the bioinformatic data suppliers 120 based on their contribution.

Accordingly, embodiments of the invention as shown in FIG. 1 can allow a plurality of unrelated bioinformatic data suppliers 120 to contribute bioinformatic data 122 to a bioinformatic data clearinghouse 110 and to be compensated for the value of the bioinformatic data 122 in generating bioinformatic data analysis results 112 that are sold to at least one bioinformatic data analysis results customer 130. Stated differently, the bioinformatic data clearinghouse 110 can procure bioinformatic data 122 from bioinformatic data suppliers 120 and provide value-added processing of the bioinformatic data in exchange for rights to royalty streams that the bioinformatic data clearinghouse 110 receives from at least one bioinformatic data analysis results customer 130 that has purchased the bioinformatic data analysis results 112 that are provided by the bioinformatic data clearinghouse 110. Thus, the bioinformatic data clearinghouse can serve as a value-added data exchange from the bioinformatic data suppliers 120 to the bioinformatic data analysis results customers 130, and can serve as a compensation broker or distributor from the bioinformatic data analysis results customers 130 back to the bioinformatic data suppliers 120.

The bioinformatic data suppliers 120 can obtain increased value for their bioinformatic data by allowing their data to be aggregated with other bioinformatic data from other bioinformatic data suppliers 120, to produce new and useful bioinformatic data analysis results 112. The bioinformatic data clearinghouse 110 can profit by selling the bioinformatic data analysis results 112 at a premium and by retaining a commission, for example a percentage of the total compensation 114 received from bioinformatic data analysis results customers 130. Finally, bioinformatic data analysis results customers 130 can obtain bioinformatic data analysis results 112 that they may not be able to generate internally or by interacting with one or a small set of bioinformatic data suppliers 120, and can simplify the compensation process by allowing the clearinghouse 110 to provide apportioned compensation 124. Incentives therefore may be provided for bioinformatic data suppliers 120 and bioinformatic data analysis results customers 130 to cooperate, share bioinformatic data 122 and produce new bioinformatic data analysis results 112. Rather than merely talking about forming a bioinformatics community, economic incentives may be provided by embodiments of the present invention, to form this community.

Still referring to FIG. 1, it will be understood that the bioinformatic data 122, bioinformatic data analysis results 112, total compensation 114 and/or apportioned compensation 124 may be transferred among the bioinformatic data suppliers 120, the bioinformatic data clearinghouse 110 and the bioinformatic data analysis results customers 130 of FIG. 1 using a network such as the Internet, other electronic media such as CD-ROMs, a telephone and/or conventional mail transfer. Accordingly, embodiments of FIG. 1 are not limited to the bioinformatic data clearinghouse 110 being electronically linked with the bioinformatic data suppliers 120 and/or the bioinformatic data analysis results customers 130. However, electronic links may facilitate efficiency, accuracy and/or speed.

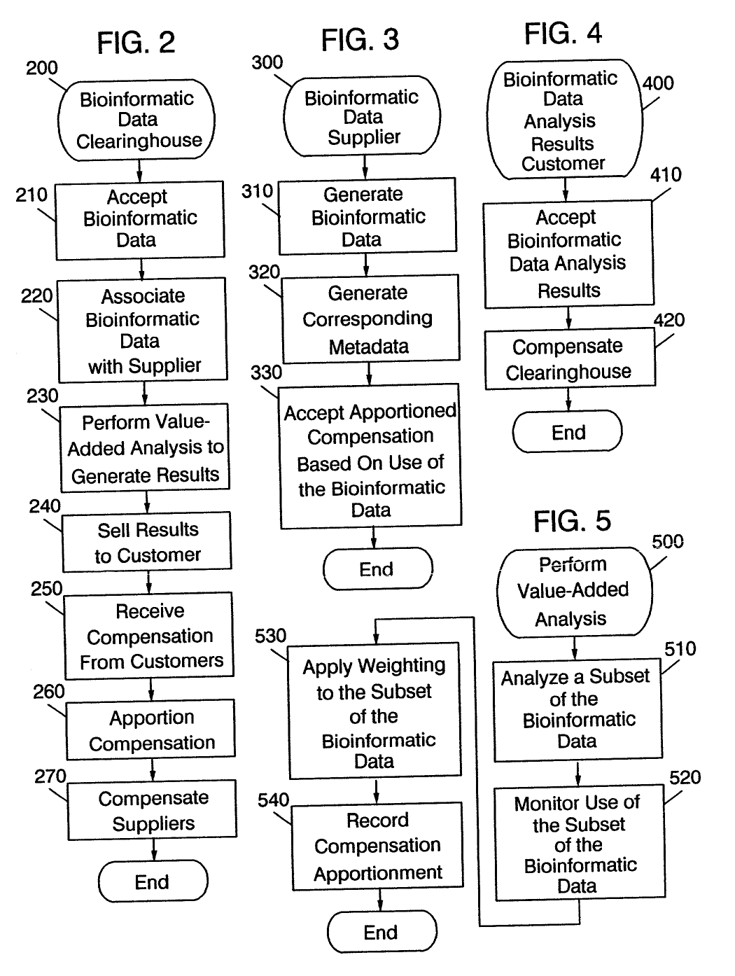

FIG. 2 is a flowchart of operations that may be performed by a bioinformatic data clearinghouse, such as the bioinformatic data clearinghouse 110 of FIG. 1, according to embodiments of the present invention. Referring to FIG. 2, these operations 200 begin by accepting bioinformatic data at Block 210. For example, bioinformatic data 122 may be accepted from corresponding bioinformatic data suppliers 120 of FIG. 1.

At Block 220, the bioinformatic data is associated with the bioinformatic data suppliers 120. For example, the bioinformatic data may be accepted as a data file and a field can be added to the data file which contains an identification of the bioinformatic data supplier 120. Alternatively, the identification may be provided in the bioinformatic data that is supplied by the bioinformatic data suppliers 120.

Thus, as shown in FIG. 6, a bioinformatic data file 600 may include a set of bioinformatic data 610, associated metadata 620 and an associated supplier ID 630. The bioinformatic metadata 620 will be described below. The bioinformatic data 610 and metadata 620 may be generated by or for bioinformatic data suppliers, such as bioinformatic data suppliers 120 of FIG. 1. The supplier ID 630 also may be generated by the bioinformatic data supplier 120 and/or by a bioinformatic data clearinghouse, such as the bioinformatic data clearinghouse 110 of FIG. 1, to thereby associate the bioinformatic data with the corresponding bioinformatic data supplier. Hierarchies of associations also may be provided where, for example, a bioinformatic datum is associated with an organization, a laboratory and/or an individual investigator.

Alternatively, the data may be accepted at Block 210 in the form of a data object. As is well known to those having skill in the art, an object defines a data structure and a set of operations or functions that can access the data structure. The data structure may be represented as a frame that includes variables or attributes of the data in the frame. Each operation or function that can access the data structure is called a “method”.

FIG. 7 illustrates an example of a bioinformatic data object 700, including a frame 740 and associated methods 750. As shown in FIG. 7, the frame 740 includes bioinformatic data 710, metadata 720 and a supplier ID 730. The bioinformatic metadata 720 will be described below. The bioinformatic data 710 and metadata 720 may be generated by or for bioinformatic data suppliers, such as bioinformatic data suppliers 120 of FIG. 1. The supplier ID 730 also may be generated by the bioinformatic data supplier and/or the bioinformatic data clearinghouse, such as the bioinformatic data clearinghouse 110 of FIG. 1, to thereby associate the bioinformatic data with the corresponding bioinformatic data supplier.

Referring now to Block 230, value-added analysis is performed by or for the bioinformatic data clearinghouse 110, to generate bioinformatic data analysis results, such as the bioinformatic data analysis results 112 of FIG. 1. Bioinformatic data analysis results 112 may be generated using bioinformatic data analysis systems and methods that are now known and/or are developed hereafter. These bioinformatic data analysis systems and methods include expression profiling, proteomics, bioinformatic data software analysis tools, image analysis tools, clustering/sorting software, self-organized maps and/or many other bioinformatic data analysis tools. A particularly useful set of value-added bioinformatic data analysis tools is described in U.S. patent application Ser. No. 09/657,218, entitled Systems, Methods and Computer Program Products for Processing Genomic Data in an Object-Oriented Environment to Wilbanks et al., filed Sep. 7, 2000, and assigned to the assignee of the present application, the disclosure of which is hereby incorporated herein by reference in its entirety.

Referring now to Block 240, the bioinformatic data analysis results 112 are sold to one or more bioinformatic data analysis results customers, such as the bioinformatic data analysis results customers 130 of FIG. 1. At Block 250, the bioinformatic data clearinghouse 110 receives compensation from the customers 130, such as the total compensation 114 of FIG. 1. It will be understood that this total compensation may be in the form of a lump sum payment, a royalty stream, securities such as corporate stock, other forms of payment and/or any other item of value, and may be pre-negotiated by or for the clearinghouse 110. Then, at Block 260, the compensation is apportioned by or for the bioinformatic data clearinghouse 110. Compensation may be apportioned so that the bioinformatic data suppliers 120 that supply the subset of the bioinformatic data that was analyzed to generate the bioinformatic data analysis results 112 that were provided are compensated for their contribution. Stated differently, the total compensation 114 may be subdivided in a pro-rata fashion based on the contribution of the bioinformatic data that is supplied by a bioinformatic data supplier relative to other bioinformatic data that is supplied by other suppliers, to generate the bioinformatic data analysis results 112. Compensation also may be divided according to hierarchy, such as an organization, laboratory and/or individual that supplied the bioinformatic data.

Finally, referring to Block 270, after compensation is apportioned, suppliers are compensated at Block 270, for example, by providing the appropriate apportioned compensation 124 of FIG. 1 to the appropriate bioinformatic data suppliers 120 of FIG. 1. The apportioned compensation that is provided to the suppliers at Block 270 may take the form of a fixed cash payment, a portion of future cash flows, securities, and/or other cash or non-cash compensation. In one embodiment, a fixed percentage of the total compensation, for example the total cash compensation 114 that is received from a bioinformatic data analysis results customer 130 in FIG. 1, will flow through the bioinformatic data clearinghouse 110 and be supplied to bioinformatic data suppliers as apportioned compensation 124. The percentage that is not supplied back to the suppliers 120 may be retained by the clearinghouse 110 as profit and/or provided to other subcontractors. In other alternatives, the clearinghouse may keep a fixed dollar amount and/or other arrangements may be provided for funding the clearinghouse 110.

Referring now to FIG. 3, operations that may be performed by bioinformatic data suppliers, such as the bioinformatic data suppliers 120 of FIG. 1, now will be described. As shown in FIG. 3, the operations 300 that are performed by the bioinformatic data suppliers begin with generating bioinformatic data by or for the bioinformatic data supplier at Block 310. Optionally, at Block 320, corresponding metadata, such as metadata 620 and 720 of FIGS. 6 and 7 respectively, also is generated.

As will be understood by those having skill in the art, metadata refers to data about data. More specifically, in genomics, the bioinformatic data may include gene expression data, data which quantifies the levels of genetic or proteomic product presence in actual organic cells and/or the like, whereas the metadata can describe the environment and/or experiment from which the expression data was obtained (organism, tissue type, organ, type of disease or healthy state, drug exposed to, etc.), the tools with which the data was obtained, the time at which the expression data was obtained (developmental stage of the cell, stage of disease, time after exposure to drug, etc.), gene and protein accession numbers, sequence, cited literal gene and protein structural features, and/or other information about the data which may be useful to the bioinformatic data clearinghouse 110 in performing data analysis. If metadata is supplied along with the bioinformatic data, then the bioinformatic data supplier 120 and/or the bioinformatic data clearinghouse 110 can associate the bioinformatic data and metadata with the supplier, for example, as was described at Block 220 of FIG. 2. Finally, at Block 330, the bioinformatic data supplier 120 accepts an apportioned compensation that is based on the use of the bioinformatic data to achieve the bioinformatic data analysis results that were provided to bioinformatic data analysis results suppliers.

FIG. 4 is a block diagram of operations that may be performed by bioinformatic data analysis results customers, such as the bioinformatic data analysis results customers 130 of FIG. 1. Referring now to FIG. 4, these operations 400 include accepting bioinformatic data analysis results, such as the bioinformatic data analysis results 112 of FIG. 1, at Block 410. It will be understood that prior to accepting the bioinformatic data analysis results at Block 410, the bioinformatic data analysis results customer 130 may commission the bioinformatic data clearinghouse 110 to obtain desired results, based on the field of business and/or desired research activities of the bioinformatic data analysis results customer 130. At Block 420, the bioinformatic data analysis results customer compensates the clearinghouse 110. As was described above, this compensation may be in the form of a lump sum, royalties, stock and/or other cash or non-cash compensation, and preferably is prearranged prior to accepting bioinformatic data analysis results at Block 410.

Referring now to FIG. 5, operations to perform value-added analysis by or for a bioinformatic data clearinghouse according to embodiments of the present invention, now will be described in detail. These operations 500 to perform value-added analysis may correspond to operations of Block 230 of FIG. 2, and may be performed by or for a bioinformatic data clearinghouse 110 of FIG. 1.

Referring again to FIG. 5, at Block 510, a subset of the bioinformatic data is analyzed. For example, the subset of the genomic data may be analyzed to obtain previously unknown specifications of biological pathways, previously unknown relationships between the expression patterns of multiple genes, gene sequences for genes that are implicated in a particular biological phenomenon, peptide sequences for proteins that may be key to a pharmaceutically interesting biological phenomena, chemical specifications of a binding site to a protein that may be key to a pharmaceutically interesting biological phenomena and/or other bioinformatic data analysis results, using known bioinformatic data analysis tools and/or bioinformatic data analysis tools that are developed in the future. It will be understood that the subset of the bioinformatic data may be preselected based on the desired bioinformatic data analysis results and/or may be selected from all the bioinformatic data by the analysis tool as it is needed.

Referring now to Block 520, during and/or after the analysis at Block 510, the use of the subset of bioinformatic data is monitored or logged. For example, the subset of the bioinformatic data that is used as inputs for the bioinformatic data analysis may be monitored or logged. More specifically, a count of the bioinformatic data files 600 and/or bioinformatic data objects 700 of FIGS. 6 and 7, respectively, that are used in bioinformatic data analysis of Block 510 may be monitored or logged. Alternatively, the bioinformatic data file 600 and/or bioinformatic data objects 700 that actually are used to generate the final bioinformatic data analysis results may be counted without counting the files and/or objects that were selected but were not used in the final results. In yet another alternative, the number of times a given bioinformatic data file 600 and/or bioinformatic data object 700 is accessed may be counted. Combinations of the above and/or other monitoring/logging techniques may be used.

Referring now to Block 530, a weighting also may be applied to the subset of the bioinformatic data. In weighting, the importance of bioinformatic data in achieving data analysis results may also be taken into account. For example, as described in a publication entitled Singular Value Decomposition for Genome—Wide Expression Data Processing and Modeling, to Alter et al., PNAS, Aug. 29, 2000, Vol. 97, No. 18, Aug. 29, 2000, pp. 10101-10106, eigengenes may be decorrelated to support references relative to other genes. Data normalization also may be used to filter the eigenvalues that are inferred to represent noise or experiential artifacts. These rating decorrelations/normalizations may be used to ascertain an importance and/or value of a supplier's bioinformatic data in the bioinformatic data analysis results, and may also be used as a factor in compensation. Finally, at Block 540, the compensation apportionment is recorded for later use in distributing the total compensation 114 that is received from a bioinformatic data analysis results customer 130 to the bioinformatic data suppliers 120.

Accordingly, embodiments of the present invention can allow commercializers of pharmacological or other products to obtain bioinformatic data analysis results that may not be available by internal development and/or by collaboration with one or a few suppliers. Bioinformatic data suppliers also can obtain enhanced value for their contribution by allowing their bioinformatic data to be aggregated with other bioinformatic data from other suppliers, to produce new bioinformatic data analysis results. Thus, suppliers who are working in related fields but are unknown to one another can obtain enhanced value for their data. Large pharmacological companies also can market collateral bioinformatic data that is not being used for internal research projects. Bioinformatic data analysis tools also can have enhanced value by allowing them to operate on many sets of bioinformatic data from many suppliers. Drug development and other beneficial results can be encouraged, so that a collaborative bioinformatics community can be formed with appropriate economic incentives.

The present invention has been described with reference to block diagrams and/or flowchart illustrations of methods and systems including computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function specified in the block diagrams and/or flowchart block or blocks.

The computer program instructions may also be loaded onto a computer or other programmable data processing apparatus to cause a series of operational steps to be performed on the computer or other programmable apparatus to produce a computer implemented method or process such that the instructions which execute on the computer or other programmable apparatus provide steps for implementing the functions specified in the block diagrams and/or flowchart block or blocks. Moreover, some or all of the operational steps need not be performed on a computer or other programmable data processing apparatus, and the series of operational steps can implement methods and/or systems of doing business.

It should also be noted that in some alternative implementations, the functions noted in the blocks may occur out of the order noted in the flowcharts. For example, two blocks shown in succession may in fact be executed substantially concurrently or the blocks may sometimes be executed in the reverse order, depending upon the functionality involved.

In the drawings and specification, there have been disclosed typical preferred embodiments of the invention and, although specific terms are employed, they are used in a generic and descriptive sense only and not for purposes of limitation, the scope of the invention being set forth in the following claims.

What is claimed is:

1. A method of processing bioinformatic data comprising:
accepting bioinformatic data from multiple corresponding bioinformatic data suppliers;
analyzing a subset of the bioinformatic data to generate bioinformatic data analysis results;
providing the bioinformatic data analysis results to at least one bioinformatic data analysis results customer; and
compensating the bioinformatic data suppliers that supplied the subset of the bioinformatic data for their supplying the subset of the bioinformatic data that was analyzed to generate the bioinformatic data analysis results that were provided to the at least one bioinformatic data analysis results customer.

2. A method according to claim 1 wherein the bioinformatic data comprises at least one of genomic data, chemical data, data on an effect of drugs or other therapies, medical patient data and information about phenotypes or disease states.

3. A method according to claim 1 wherein the bioinformatic data analysis results comprise at least one of specifications of biological pathways, relationships between expression patterns of multiple genes, gene sequences for genes that are related in a particular biological phenomenon, gene sequences with homology to genes of unknown function, peptide sequences for proteins that are related to a biological phenomena, peptide sequences with homology to proteins of unknown function, a chemical specification of a binding site to a protein that is related to a biological phenomena, a toxicity profile of a therapeutic chemical, and a chemical specification of a therapeutic chemical.

4. A method according to claim 1 wherein the bioinformatic data suppliers comprise at least one of a pharmaceutical company research and development lab, an expression analysis outsourcer, a genome sequencing searcher and an academic research lab.

5. A method according to claim 1 wherein the bioinformatic data analysis results customers comprise at least one of a pharmaceutical company, a drug development company, an academic laboratory, a medical doctor and a genetic counselor.

6. A method according to claim 1 wherein the accepting is preceded by generating the bioinformatic data at the corresponding bioinformatic data suppliers.

7. A method according to claim 6:
wherein the generating further comprises generating metadata corresponding to the bioinformatic data at the corresponding bioinformatic data suppliers; and
wherein the accepting comprises accepting the bioinformatic data and the corresponding metadata from the corresponding bioinformatic data suppliers.

8. A method according to claim 7 wherein the metadata comprises at least one of a description of a cell from which the associated bioinformatic data was generated, a description of an environment from which the associated bioinformatic data was generated, a description of a tool and/or experimental protocol that was used to generate the bioinformatic data, a description of a time at which the associated bioinformatic data was generated, a description of a chemical with which a subject of the bioinformatic data was treated, and a description of a pre-treatment state of the subject of the bioinformatic data.

9. A method according to claim 1 wherein the accepting is followed by:
associating the bioinformatic data with the corresponding bioinformatic data suppliers.

10. A method according to claim 1:
wherein the analyzing is preceded by identifying the subset of the bioinformatic data from the bioinformatic data; and
wherein the analyzing is followed by recording the identification of the subset of the bioinformatic data that was used in the analyzing.

11. A method according to claim 1 wherein the analyzing comprises at least one of expression profiling, proteomic analysis, image analysis, clustering, sorting and generating a self-organized map.

12. A method according to claim 1 wherein the providing comprises selling the bioinformatic data analysis results to the at least one bioinformatic data analysis results customer for a lump sum payment, a royalty stream, and/or securities, such as corporate stock.

13. A method according to claim 12 wherein the compensating comprises providing the bioinformatic data suppliers that supplied the subset of the bioinformatic data with a portion of the lump sum payment, the royalty stream and/or securities, such as corporate stock, as compensation for their supplying the subset of the bioinformatic data that was analyzed to generate the bioinformatic data analysis results that were sold to the at least one bioinformatic data analysis results customer.

14. A method according to claim 1:
wherein the analyzing further comprises:
identifying the subset of the bioinformatic data from the bioinformatic data; and
determining a relative contribution of members of the subset of the bioinformatic data to the bioinformatic data analysis results.

15. A method according to claim 14 wherein the compensating comprises compensating the bioinformatic data suppliers that supplied the subset of the bioinformatic data for their supplying the subset of the bioinformatic data as a function of a relative contribution of the members of the subset of the bioinformatic data to the bioinformatic data analysis results.

16. A method of providing bioinformatic data comprising:
supplying bioinformatic data to a bioinformatic data clearinghouse; and
accepting compensation from the bioinformatic data clearinghouse for the bioinformatic data, wherein the compensation is a function of use of the bioinformatic data by the bioinformatic data clearinghouse to generate bioinformatic data analysis results that are provided to at least one bioinformatic data analysis results customer, relative to use of other bioinformatic data by the bioinformatic data clearinghouse to generate the bioinformatic data analysis results that are provided to the at least one bioinformatic data analysis results customer.

17. A method according to claim 16 wherein the bioinformatic data comprises at least one of genomic data, chemical data, data on an effect of drugs or other therapies, medical patient data and information about phenotypes or disease states.

18. A method according to claim 16 wherein the bioinformatic data analysis results comprise at least one of specifications of biological pathways, relationships between expression patterns of multiple genes, gene sequences for genes that are related in a particular biological phenomenon, gene sequences with homology to genes of unknown function, peptide sequences for proteins that are related to a biological phenomena, peptide sequences with homology to proteins of unknown function, a chemical specification of a binding site to a protein that is related to a biological phenomena, a toxicity profile of a therapeutic chemical, and a chemical specification of a therapeutic chemical.

19. A method according to claim 16 wherein the supplying and accepting are performed by at least one of a pharmaceutical company research and development lab, an expression analysis outsourcer, a genome sequencing researcher and an academic research lab.

20. A method according to claim 16 wherein the bioinformatic data analysis results customers comprise at least one of a pharmaceutical company, a drug development company, an academic laboratory, a medical doctor and a genetic counselor.

21. A method according to claim 16 wherein the supplying is preceded by generating the bioinformatic data.

22. A method according to claim 21:
wherein the generating further comprises generating metadata corresponding to the bioinformatic data; and
wherein the supplying comprises supplying the bioinformatic data and the corresponding metadata.

23. A method according to claim 22 wherein the metadata comprises at least one of a description of a cell from which the associated bioinformatic data was generated, a description of an environment from which the associated bioinformatic data was generated, a description of a tool and/or experimental protocol that was used to generate the bioinformatic data, a description of a time at which the associated bioinformatic data was generated, a description of a chemical with which a subject of the bioinformatic data was treated, and a description of a pretreatment state of the subject of the bioinformatic data.

24. A system for processing bioinformatic data comprising:
a plurality of bioinformatic data suppliers;
at least one bioinformatic data analysis results customer; and
a bioinformatic data clearinghouse that is configured to accept bioinformatic data from the plurality of bioinformatic data suppliers, to analyze a subset of bioinformatic data to generate bioinformatic data analysis results, to provide the bioinformatic data analysis results to the at least one bioinformatic data analysis results customer and to compensate the bioinformatic data suppliers that supplied the subset of bioinformatic data for their supplying the subset of bioinformatic data that was analyzed to generate the bioinformatic data analysis results that were provided to the at least one bioinformatic data analysis results customer.

25. A system according to claim 24 wherein the bioinformatic data comprises at least one of genomic data, chemical data, data on an effect of drugs or other therapies, medical patient data and information about phenotypes or disease states.

26. A system according to claim 24 wherein the bioinformatic data analysis results comprise at least one of specifications of biological pathways, relationships between expression patterns of multiple genes, gene sequences for genes that are related in a particular biological phenomenon, gene sequences with homology to genes of unknown function, peptide sequences for proteins that are related to a biological phenomena, peptide sequences with homology to proteins of unknown function, a chemical specification of a binding site to a protein that is related to a biological phenomena, a toxicity profile of a therapeutic chemical, and a chemical specification of a therapeutic chemical.

27. A system according to claim 24 wherein the bioinformatic data suppliers comprise at least one of a pharmaceutical company research and development lab, an expression analysis outsourcer, a genome sequencing researcher and an academic research lab.

28. A system according to claim 24 wherein the bioinformatic data analysis results customers comprise at least one of a pharmaceutical company, a drug development company, an academic laboratory, a medical doctor and a genetic counselor.

29. A system according to claim 24 wherein the plurality of bioinformatic data suppliers are configured to generate the bioinformatic data.

30. A system according to claim 29:
wherein the plurality of bioinformatic data suppliers are configured to generate metadata corresponding to the bioinformatic data; and
wherein the bioinformatic data clearinghouse is configured to accept the bioinformatic data and the corresponding metadata from the plurality of bioinformatic data suppliers.

31. A system according to claim 30 wherein the metadata comprises at least one of a description of a cell from which the associated bioinformatic data was generated, a description of an environment from which the associated bioinformatic data was generated, a description of a tool and/or experimental protocol that was used to generate the bioinformatic data and a description of a time at which the associated bioinformatic data was generated, a description of a chemical with which a subject of the bioinformatic data was treated, and a description of a pre-treatment state of the subject of the bioinformatic data.

32. A system according to claim 24 wherein the bioinformatic data clearinghouse is further configured to associate the bioinformatic data with the corresponding bioinformatic data suppliers.

33. A system according to claim 24:
wherein the bioinformatic data clearinghouse is further configured to identify the subset of the bioinformatic data from the bioinformatic data, and to record the identification of the subset of the bioinformatic data that was used in the analyzing.

34. A system according to claim 24 wherein the analyzing comprises at least one of expression profiling, proteomic analysis, image analysis, clustering, sorting and generating a self-organized map.

35. A system according to claim 24 wherein the bioinformatic data clearinghouse is further configured to provide the bioinformatic data analysis results to the at least one bioinformatic data analysis results customer for a lump sum payment, a royalty stream, and/or securities, such as corporate stock.

36. A system according to claim 35 wherein the bioinformatic data clearinghouse is further configured to provide the bioinformatic data suppliers that supplied the subset of the bioinformatic data with a portion of the lump sum payment, the royalty stream and/or the securities as compensation for their supplying the subset of the bioinformatic data that was analyzed to generate the bioinformatic data analysis results that were provided to the at least one bioinformatic data analysis results customer.

37. A system according to claim 24 wherein the bioinformatic data clearinghouse is further configured to identify the subset of the bioinformatic data from the bioinformatic data, and to determine a relative contribution of members of the subset of the bioinformatic data to the bioinformatic data analysis results.

38. A system according to claim 37 wherein the bioinformatic data clearinghouse is further configured to compensate the bioinformatic data suppliers that supplied the subset of the bioinformatic data for their supplying the subset of the bioinformatic data as a function of a relative contribution of the members of the subset of the bioinformatic data to the bioinformatic data analysis results.

39. A bioinformatic data clearinghouse comprising:
means for accepting bioinformatic data from a plurality of bioinformatic data suppliers;
means for analyzing a subset of the bioinformatic data to generate bioinformatic data analysis results;
means for providing the bioinformatic data analysis results to at least one bioinformatic data analysis results customer; and
means for compensating the bioinformatic data suppliers that supplied the subset of the bioinformatic data for their supplying the subset of the bioinformatic data that was analyzed to generate the bioinformatic data analysis results that were provided to the at least one bioinformatic data analysis results customer.

40. A clearinghouse according to claim 39 wherein the bioinformatic data comprises at least one of genomic data, chemical data, data on an effect of drugs or other therapies, medical patient data and information about phenotypes or disease states.

41. A clearinghouse according to claim 39 wherein the bioinformatic data analysis results comprise at least one of specifications of biological pathways, relationships between expression patterns of multiple genes, gene sequences for genes that are related in a particular biological phenomenon, gene sequences with homology to genes of unknown function, peptide sequences for proteins that are related to a biological phenomena, peptide sequences with homology to proteins of unknown function, a chemical specification of a binding site to a protein that is related to a biological phenomena, a toxicity profile of a therapeutic chemical, and a chemical specification of a therapeutic chemical.

42. A clearinghouse according to claim 39 wherein the bioinformatic data suppliers comprise at least one of a pharmaceutical company research and development lab, an expression analysis outsourcer, a genome sequencing researcher and an academic research lab.

43. A clearinghouse according to claim 39 wherein the bioinformatic data analysis results customers comprise at least one of a pharmaceutical company, a drug development company, an academic laboratory, a medical doctor and a genetic counselor.

44. A clearinghouse according to claim 39 wherein the means for accepting comprises means for accepting the bioinformatic data and corresponding metadata from the corresponding bioinformatic data suppliers.

45. A clearinghouse according to claim 44 wherein the metadata comprises at least one of a description of a cell from which the associated bioinformatic data was generated, a description of an environment from which the associated bioinformatic data was generated, a description of a tool and/or experimental protocol that was used to generate the bioinformatic data, a description of a time at which the associated bioinformatic data was generated, a description of a chemical with which a subject of the bioinformatic data was treated, and a description of a pre-treatment state of the subject of the bioinformatic data.

46. A clearinghouse according to claim 39 further comprising means for associating the bioinformatic data with the corresponding bioinformatic data suppliers.

47. A clearinghouse according to claim 39 further comprising:
means for identifying the subset of the bioinformatic data from the bioinformatic data; and
means for recording the identification of the subset of the bioinformatic data that was used in the analyzing.

48. A clearinghouse according to claim 39 wherein the means for analyzing comprises means for performing at least one of expression profiling, proteomic analysis, image analysis, clustering, sorting and generating a self-organized map.

49. A clearinghouse according to claim 39 wherein the means for providing comprises means for selling the bioinformatic data analysis results to the at least one bioinformatic data analysis results customer for a lump sum payment, a royalty stream, and/or securities, such as corporate stock.

50. A clearinghouse according to claim 49 wherein the means for compensating comprises means for providing the bioinformatic data suppliers that supplied the subset of the bioinformatic data with a portion of the lump sum payment and/or the royalty stream as compensation for their supplying the subset of the bioinformatic data that was analyzed to generate the bioinformatic data analysis results that were sold to the at least one bioinformatic data analysis results customer.

51. A clearinghouse according to claim 39 further comprising:
means for identifying the subset of the bioinformatic data from the bioinformatic data; and
means for determining a relative contribution of members of the subset of the bioinformatic data to the bioinformatic data analysis results.

52. A clearinghouse according to claim 51 wherein the means for compensating comprises means for compensating the bioinformatic data suppliers that supplied the subset of the bioinformatic data for their supplying the subset of the bioinformatic data as a function of a relative contribution of the members of the subset of the bioinformatic data to the bioinformatic data analysis results.

53. A computer program product that processes bioinformatic data, the computer program product comprising a computer-usable storage medium having computer-readable program code embodied in the medium, the computer-readable program code comprising:
computer-readable program code that is configured to accept bioinformatic data from a plurality of bioinformatic data suppliers;
computer-readable program code that is configured to analyze a subset of the bioinformatic data to generate bioinformatic data analysis results;
computer-readable program code that is configured to provide the bioinformatic data analysis results to at least one bioinformatic data analysis results customer; and
computer-readable program code that is configured to authorize compensation for the bioinformatic data suppliers that supplied the subset of the bioinformatic data for their supplying the subset of the bioinformatic data that was analyzed to generate the bioinformatic data analysis results that were provided to the at least one bioinformatic data analysis results customer.

54. A computer program product according to claim 53 wherein the bioinformatic data comprises at least one of genomic data, chemical data, data on an effect of drugs or other therapies, medical patient data and information about phenotypes or disease states.

55. A computer program product according to claim 53 wherein the bioinformatic data analysis results comprise at least one of specifications of biological pathways, relationships between expression patterns of multiple genes, gene sequences for genes that are related in a particular biological phenomenon, gene sequences with homology to genes of unknown function, peptide sequences for proteins that are related to a biological phenomena, peptide sequences with homology to proteins of unknown function, a chemical specification of a binding site to a protein that is related to a biological phenomena, a toxicity profile of a therapeutic chemical, and a chemical specification of a therapeutic chemical.

56. A computer program product according to claim 53 wherein the bioinformatic data suppliers comprise at least one of a pharmaceutical company research and development lab, an expression analysis outsourcer, a genome sequencing researcher and an academic research lab.

57. A computer program product according to claim 53 wherein the bioinformatic data analysis results customers comprise at least one of a pharmaceutical company, a drug development company, an academic laboratory, a medical doctor and a genetic counselor.

58. A computer program product according to claim 53 wherein the computer-readable program code that is configured to accept comprises computer-readable program code that is configured to accept the bioinformatic data and corresponding metadata from the corresponding bioinformatic data suppliers.

59. A computer program product according to claim 58 wherein the metadata comprises at least one of a description of a cell from which the associated bioinformatic data was generated, a description of an environment from which the associated bioinformatic data was generated, a description of a tool and/or experimental protocol that was used to generate the bioinformatic data, a description of a time at which the associated bioinformatic data was generated, a description of a chemical with which a subject of the bioinformatic data was treated, and a description of a pre-treatment state of the subject of the bioinformatic data.

60. A computer program product according to claim 53 further comprising computer-readable program code that is configured to associate the bioinformatic data with the corresponding bioinformatic data suppliers.

61. A computer program product according to claim 53 further comprising:
computer-readable program code that is configured to identify the subset of the bioinformatic data from the bioinformatic data; and
computer-readable program code that is configured to record the identification of the subset of the bioinformatic data that was used in the analyzing.

62. A computer program product according to claim 53 wherein the computer-readable program code that is configured to analyze comprises computer-readable program code that is configured to perform at least one of expression profiling, proteomic analysis, image analysis, clustering, sorting and generating a self-organized map.

63. A computer program product according to claim 53 wherein the computer-readable program code that is configured to provide comprises computer-readable program code that is configured to authorize selling the bioinformatic data analysis results to the at least one bioinformatic data analysis results customer for a lump sum payment, a royalty stream, and/or securities, such as corporate stock.

64. A computer program product according to claim 63 wherein the computer-readable program code that is configured to compensate comprises computer-readable program code that is configured to provide the bioinformatic data suppliers that supplied the subset of the bioinformatic data with a portion of the lump sum payment, the royalty stream and/or the securities as compensation for their supplying the subset of the bioinformatic data that was analyzed to generate the bioinformatic data analysis results that were sold to the at least one bioinformatic data analysis results customer.

65. A computer program product according to claim 53 further comprising:
computer-readable program code that is configured to identify the subset of the bioinformatic data from the bioinformatic data; and
computer-readable program code that is configured to determine a relative contribution of members of the subset of the bioinformatic data to the bioinformatic data analysis results.

66. A computer program product according to claim 65 wherein the computer-readable program code that is configured to authorize compensation comprises computer-readable program code that is configured to authorize compensation for the bioinformatic data suppliers that supplied the subset of the bioinformatic data for their supplying the subset of the bioinformatic data as a function of a relative contribution of the members of the subset of the bioinformatic data to the bioinformatic data analysis results.
